(19) United States
(12) Patent Application Publication
(10) Pub. No.: US 2023/0389983 A1
(43) Pub. Date: Dec. 7, 2023

(54) SPRING-LOADED CATHETER FOR AN ELECTROPHYSIOLOGY (EP) STUDY AND IRREVERSIBLE ELECTROPORATION WITHIN THE HEART

(71) Applicant: Piotr FUTYMA, Rzeszow (PL)

(72) Inventor: Piotr FUTYMA, Rzeszow (PL)

(21) Appl. No.: 18/247,647

(22) PCT Filed: Sep. 28, 2021
(86) PCT No.: PCT/PL2021/000070

§ 371 Date: Apr. 3, 2023

(30) Foreign Application Priority Data
Oct. 6, 2020 (PL) P.435591

(51) Int. Cl. A61B 18/14

(52) U.S. Cl. CPC A61B 18/1492; A61B 2018/0016

(57) ABSTRACT

A spring-loaded catheter for electrophysiology studies and irreversible electroporation within the heart, comprises core of catheter protruding from sleeve main conduit is made of shape-retaining metal alloy and is bent in shape of conical spiral with different number of coils, at least one of which is equipped with sleeve electrodes imposed on this core, supply through insulated electric wires and separated from each other with plastic ring elements, where diameter Ø1 of first coil of spiral is 5 mm to 30 mm, and diameter Ø2 of last coil of spiral is 10 mm to 31 mm, while length of each of these electrodes is 2 mm to 4 mm, and diameter Ø is from 1 mm to 3 mm, and these electrodes send pulse with amplitude of 100-3000V in time and 5 microseconds to 6 milliseconds, and number of electrodes distributed on spiral of catheter ranges 10 to 65 pieces.

### BACKGROUND OF THE INVENTION

The subject of the invention is a spring-loaded catheter for an electrophysiology (EP) study and irreversible electroporation within the heart, used both to conduct electrophysiology studies (EPS) allowing for a precise assessment of the type of heart rhythm disturbances and their source in the heart muscle in people with suspicion of these disorders and with diagnosed arrhythmia and electroporation in the heart using high voltage with the ability to read signals before and after ablation and mapping, and the catheter can work with many platforms of electrophysiology systems, 3D mapping systems and pulse generators.

Procedures for treating cardiac arrhythmias include disrupting the areas causing arrhythmia by ablating the heart muscle tissue with electrical energy, which is usually accomplished by applying an alternating current, usually radiofrequency, to one or more ablation electrodes of the power necessary to alter the target tissue. Typically, these electrodes are mounted on the distal tip or portion of an invasive probe or catheter that is inserted into the patient's heart through blood vessels, especially through the femoral vein.

### DESCRIPTION OF THE PRIOR ART

The prior art cited below indicate that the treatment of cardiac arrhythmias including electrophysiology studies of ablation and cardiac mapping uses catheters with catheters probes, namely:

The electrophysiological catheter known from European patent EP2269505A1 comprises an elongated body having an elastically deformed distal region predisposed to take a spring-like shape and a first group of a plurality of electrodes disposed thereon. Each of the first of electrodes comprises an electrically active area limited to the inner surface of the spiral for use in non-contact electrophysiology studies. The second set of electrodes may also be disposed in a distal region including alternately interleaved with the first set of electrodes, each of the second electrodes having an electrically active region extending to an outer surface of the spring shape for use in contact electrophysiology studies. The distal region may be deformed into a simple configuration to be inserted into and navigated through the patient's vasculature, such as with a guide tube, where the distal region extends beyond the distal end of the introducer to form a spiral. Moreover, the electrophysiological catheter comprises a shape memory material extending through the distal (spring) region of its body, the memory material being a metal wire, part of which is enclosed in a polymer tube, the rear end of which is placed in a tubular guide (introducer).

From the international patent application WO02089687A1 there is known a catheter assembly for treating cardiac arrhythmias which comprises a catheter body and an ablative energy source. The catheter body includes a proximal portion, an intermediate portion, and a distal portion, the intermediate portion extending from the proximal portion and defining the longitudinal axis, and the distal portion extending from the intermediate portion and including an ablative section and an end. The ablative section forms a loop that defines a diameter larger than the outer dimension of the entrance to the pulmonary vein. The end extends distally from the ablation section and is configured to locate the pulmonary vein. Finally, the ablative energy source is associated with the ablation section. In the configuration, upon activation of the energy source, the ablation section ablates the desired lesion pattern. In one preferred embodiment, the ablation section forms a distally tapering spiral, while the end includes a relatively linear guide section. In the preferred configuration, the end readily locates the pulmonary vein and guides the ablative segment to a position around the outlet of the pulmonary vein.

In turn, the international patent application WO2019089199A1 discloses a method of cardiac catheterization by means of a spring-loaded catheter containing a flexible, electrically insulated tube and a plurality of ablation electrodes placed on the outer surface of the electrically insulated tube and a plurality of microelectrodes also electrically insulated from each other and from the ablation electrodes. Moreover, the catheter includes a retention element and a shape memory that forces it to form spring loops. In addition, the method of the invention includes also reading bioelectric signals from the heart with microelectrodes and conducting electricity through selected ablation electrodes to induce damage to the ventricle, and taking bioelectric readings from selected microelectrodes and mapping the electrical activity in the heart from these readings. Catheterization is accomplished by inserting the catheter into the heart, sliding the catheter through the sheath that surrounds the multi-electrode probe into the ventricle. The sheath is retracted to expose the probe. As the sheath is retracted, the exposed probe expands into a helical configuration and the electrodes contact the ventricular endocardial surface at multiple points of contact.

From the US patent application U.S. Pat. No. 5,374,287A there is known a defibrillator and a stimulator catheter comprising a flexible, electrically non-conductive probe having an electrically conductive path lengthwise therein. To the one end of the probe there is attached a defibrillation electrode capable of anchoring the probe to the septum of the heart and of transmitting a portion of the electrical defibrillation pulse sufficient to defibrillate the heart from said conductive path directly to the interior of the septum. The defibrillation pulse is delivered in such way to avoid damage to the heart tissue immediately adjacent to the defibrillator electrode. In a preferred embodiment, the defibrillator electrode is spiraled; however, it is also predicted to be a lance. Alternatively, the catheter further comprises a ground electrode, an on-demand stimulator electrode, and an additional defibrillator electrode attached to the probe.

Also, from US patent application U.S. Pat. No. 5,133,365A, there is known a modified cardiac electrode adapted for use with an automatic implantable cardioverter/defibrillator (AICD), consisting of an elongated, flexible, tubular plastic catheter body that is preformed such that when deformed takes the shape of a tapering spiral or spiral. The catheter body supports a defibrillating electrode attached to the outer wall of the catheter body and is connected by a suitable cable to the proximal connector to conform to the AICD pulse generator. The enhanced probe also includes an end electrode to detect cardiac activity and provide information to the AICD pulse generator to monitor its operation. The probe of the present invention is designed to seat the endocardium with predominantly right ventricular electrode structures and provides a significantly increased electrode surface area in contact with the heart tissue, thereby maximizing the energy delivered to the heart during defibrillation.

From the US patent application US2004181160A1 there is known a system based on a non-extensible, non-contact, multi-electrode miniature catheter, which is used for measuring of electrical potentials in the heart cavity and for electrophysiological mapping of the heart. The system includes a non-contact multi-electrode catheter probe that can be inserted into the heart cavity filled with blood without obstructing it. The probe for measuring the electrical potentials in the heart cavity comprises: a multi-electrode end portion conforming to the shape of a cylindrical spiral that is positioned not to contact the endocardial surface of the heart and is inserted transdermally into the heart cavity.

Also from the Polish patent specification PL227730B1 there is known an ablation-mapping catheter used for electrocardiological procedures, containing at least eight diagnostic rings, connected via connectors to the generator(s), which enable non-fluoroscopic mapping in a three-dimensional electroanatomical system, with the diagnostic rings evenly spaced at the distal end of the electrode. The ablation mapping catheter has a control handle, a straight main cable, a distal end ring mounted on it, and diagnostic rings including distal and proximal ones, and two wiring harnesses connecting these diagnostic rings and an end distal ring with the electrophysiology system. In addition, the catheter is made of an elastic material that allows it to be bent easily, and the distal end is equipped with a control system located in the handle of the catheter and connected by appropriate tendons, the catheter being inserted into peripheral venous or arterial vessels (femoral vein/artery) of the patient and then is guided through the main vessels into the right or left chambers of the heart. The control system of the catheter allows its rounded distal end to be bent.

In the commonly known catheters used for electrophysiology studies and heart mapping there is a small number of electrodes with relatively small dimensions, which means that during electroporation that is carried out with them, gas is generated, bubbles of which can enter the brain, which threatens the patient's life and health, and in addition, may occur punctures (plasma is generated) or barotrauma.

The aim of the invention is to develop such structure of a spring-loaded catheter for electrophysiology studies, which will also allow to carry out a patient-safe, irreversible electroporation of the heart tissue using high-amplitude electrical pulses, as a result of which the heart cells die as a result of destabilization of the cell membrane.

The spring-loaded catheter for an electrophysiology (EP) study and irreversible electroporation within the heart according to the invention is characterized in that the core protruding from the sleeve main conduit of the catheter is made of a metal alloy that retains shape memory and is bent in the shape of a conical spiral with a varying number of coils, at least one of which is equipped with sleeve electrodes placed on the core, powered by insulated electric wires and separated from each other by plastic ring elements, the diameter Ø1 of the first coil of the spiral ranges from 5 mm to 30 mm, and the diameter Ø2 of the last coil of the spiral ranges from 10 mm to 31 mm, while the length of each of these electrodes is from 2 mm to 4 mm, and their diameter Ø is from 1 mm to 3 mm, and these electrodes transmit a pulse with an amplitude of 100-3000V in time and from 5 microseconds to 6 milliseconds, and the number of electrodes distributed on the spiral of the catheter ranges from 10 to 65 pieces.

Preferably, the conical spiral of the catheter is a converging spiral or a divergent spiral.

It is also preferable that the maximum number of coils of the spiral in the catheter is 5 coils and the number of sleeve electrodes arranged on the coils of the spiral is 65.

It is also preferable if the two terminal coils of the conical spiral have 15 sleeve electrodes separated by plastic ring elements, and the central coil of the spiral is covered with a plastic sheath covering the catheter core together with electric conductors supplying current to the sleeve electrodes of the last coil.

It is also preferable if a three-part sheath is slidably placed on the sleeve main conduit, the two terminal parts of which are conductive sheaths, and the third sheath placed between them is made of insulating material, and the conductive sheaths are made entirely of electrically conductive material or half of them are made of electrically conductive material and half of insulating material, or ¼ of these sheaths are made of electrically conductive material, and ¾ of insulating material, where the electrically conductive material is copper or a copper alloy.

It is also preferable when a PTFE coated stainless steel stabilizing bar is placed in the sleeve main conduit and when the stabilizing bar exits the main conduit through the opening in front of the conical spiral so that the spiral is wound on the main conduit, or when the stabilizing bar that is placed in the sleeve main conduit passes through the holes of the sleeve electrodes and the holes of the plastic ring elements of the conical spiral of the catheter.

It is also preferable if the catheter ends with a sleeve electrode or a plastic ring element.

It is also preferable that a driver handle is provided at the rear end of the sleeve main conduit in front of the electrically connected connector for bending the end of the catheter spiral only. It is also preferable if the sleeve electrodes are provided with thermistors or are provided with thermocouples.

It is also preferable to made the sleeve electrodes entirely of electrically conductive material, or to made the electrodes of electrically conductive material in half of their diameters and half of electrically non-conductive material, or they are made of electrically conductive material in ¼ of their diameters, and in the remainder ¾ of electrically non-conductive material, the electrically conductive material of these sleeve electrodes is platinum, gold or surgical steel, and the electrically non-conducting material is PVC or Teflon.

It is also preferable if the core of the catheter is made of nitinol and covered with a plastic coating.

It is also preferable if the number of pins placed in the connector of the catheter corresponds to the number of electric wires supplying the sleeve electrodes and the number of sensors placed in these electrodes.

Pre-clinical test of the spring-loaded catheter according to the invention have shown that the use of a large number of electrodes transmitting high amplitude pulses causes that the catheter provides energy much higher than any available and currently commonly used catheters of this type, which minimizes the occurrence of unforeseen life and health threatening situations during the procedure, and the catheter:
after slipping out of the vascular sheath, it aims to obtain the optimal spring shape;
adapts to the shape of the surface in which it is located depending on the individual anatomical conditions of the heart in different patients;
has the ability to work with many platforms, which minimizes the limitations related to the availability of “only” and “specific” cooperating equipment.

The spring-loaded catheter according to the invention is a universal solution that can be used both for electrophysiology studies and cardiac mapping as well as for electroporation procedures in many configurations, especially such as: single- or two-electrode electroporation, single-electrode-interregnal electroporation, etc., and its simple and flexible structure is significantly minimizes the risk of heart perforation, while the materials used for the construction of the electrodes are relatively easily accessible, which greatly facilitates their production, and the fact that the core of the catheter is made of nitinol allows to remember its original shape and restore it under the influence of appropriate external conditions (for example, changes in the magnetic field or temperature). On the other hand, in a preferred embodiment of the catheter according to the invention, the use of a sliding three-part sheath enables the maximization of the electrically active electrode surface through which electroporation pulses are delivered, which minimizes the risk of complications such as the occurrence of punctures, barotrauma or gas bubbles, and the end of the catheter with a plastic ring element minimizes the risk of mechanical traumatization of tissue. In addition, providing the electrodes with sensors such as thermistors and thermocouples allows to control the temperature of these electrodes, which may increase with some pulse configurations.

### BRIEF DESCRIPTION OF FIGURES

The subject of the invention in eight variants of its implementation is shown in FIGS. 1-31, in which FIG. 1-7 show a first embodiment of a spring-loaded catheter for electrophysiology and cardiac irreversible electroporation having three coils with a converging spiral profile at the front end;

FIG. 1 is a top view of the first embodiment of catheter;

FIG. 2 is a front view of a spring-loaded catheter of this embodiment;

FIG. 3 is a cross sectional view of the catheter main conduit along A-A line;

FIG. 4 is the same first embodiment of the catheter with a perspective view of the coils from the posterior and lateral sides;

FIG. 5—the same first embodiment of the catheter in the side view from its connector side;

FIG. 6—the same first embodiment of the catheter in a perspective view with its coils from the anterior side and from above;

FIG. 7—enlarged “B” detail of the anterior part of the three-coil catheter;

FIGS. 8-10 show a second embodiment of a spring-loaded catheter for electrophysiology studies and cardiac irreversible electroporation with two coils with a divergent spiral profile at its front end, whereby

FIG. 8 is a perspective view of the second embodiment of the spring-loaded catheter with the coils seen from the posterior and lateral sides;

FIG. 9—the spring-loaded catheter in a front view;

FIG. 10—the main conduit of the catheter in a cross-section along the C-C line;

FIGS. 11-13 show a third embodiment of a spring-loaded catheter for electrophysiology and cardiac irreversible electroporation having five coils with a divergent spiral profile at its front end, while

FIG. 11 shows the same third embodiment of the spring-loaded catheter with a side and back view of the coils;

FIG. 12—the spring-loaded catheter in front view;

FIG. 13—main conduit of the catheter in cross-section along the D-D line;

FIGS. 14-16—show a fourth embodiment of the spring-loaded catheter for electrophysiology study and cardiac irreversible electroporation with three coils at its front end with a profile of a convergent spiral, the middle of which is a plastic coil devoid of annular electrodes, while

FIG. 14 shows the same fourth embodiment of the spring-loaded catheter with a perspective view of the coils from the side and back in a perspective view;

FIG. 15—the front view of the spring-loaded catheter;

FIG. 16—the main conduit of the catheter in cross-section along the E-E line;

FIGS. 17-21 show a fifth embodiment of a spring-loaded catheter for electrophysiology study and cardiac irreversible electroporation having at its front end an incomplete coil with a profile forming part of a converging spiral and at the other end a controller equipped with a handle and an electrical connector;

FIG. 17 is a perspective view of a spring-loaded catheter according to this embodiment;

FIG. 18 is a front view of the same spring-loaded catheter;

FIG. 19 is a cross section of the main catheter conduit along the F-F line;

FIG. 20—the same fifth embodiment of the catheter in a view from its connector side;

FIG. 21—enlarged “G” detail of the spring end of the catheter;

FIGS. 22-28 show the sixth embodiment of the spring-loaded catheter for electrophysiology study and cardiac irreversible electroporation with three coils with a converging spiral profile at its end, while

FIG. 22 shows a spring-loaded catheter according to this embodiment, on the main conduit of which several conductive sheaths are mounted, separated from each other by insulating sleeve sheaths in a perspective view;

FIG. 23—the same catheter after sliding on the coils of its spiral conductive sleeve sheaths and a sleeve insulating sheath mounted on the middle coil in the perspective view;

FIG. 24—the same, sixth embodiment of catheter in the side view from its connection;

FIG. 25—a triple coil of the same catheter in a vertical section along the H-H line;

FIG. 26—enlarged “J” detail sheaths of the coils of the catheter spiral in section along the H-H line, representing the first embodiment thereof in FIG. 24;

FIG. 27—the same enlarged “J” detail of the sheath of one of the coils of the catheter spiral in section along the line H-H in FIG. 24, constituting the second embodiment of its implementation, and

FIG. 28—the same enlarged “J” detail of the sheath of one of the coils of the catheter spiral in section along the H-H line, in FIG. 24, a third embodiment thereof,

FIG. 29—shows a seventh embodiment of the spring-loaded catheter for electrophysiology study and cardiac irreversible electroporation having at its front end three coils with a convergent spiral profile wound on the main conduit of the catheter, additionally equipped with a stabilizing rod partially located in the conduit, in front view;

FIG. 30—three coils of the catheter spiral wound on the main conduit according to the seventh variant its execution along the K-K line;

FIG. 31 shows the eighth embodiment of the spring-loaded catheter for electrophysiology studies and irreversible electroporation of the heart, having at its front end three coils with a spiral profile converging with the main stabilizing bar placed additionally in them and in the conduit in the front view;

FIG. 32 is a front view of an embodiment of one of the plurality of annular electrodes provided with a conductor for electric current and a thermistor;

FIG. 33 is an axial section view of the same annular electrode along the L-L line, front view of one of the plurality of ring electrodes;

FIG. 34 shows an example of manufacturing one of many ring electrode from front view

FIG. 35—the same electrode cross-section along the M-M line made of a homogeneous electrically conductive material;

FIG. 36—the same electrode cross-section along the M-M line, where one half of it is made of electrically conductive material and the other half is made of insulating material;

FIG. 37—the same electrode in the cross-section along the M-M line, where ¾ of the electrode is made of insulating material, and ¾ of conductive material;

FIGS. 38-39—show a simplified example of the adaptation of the spiral profile of the catheter to a flat or concave side view of the surface of the heart cavity during the procedure, and

FIG. 40—shows an example of insertion of a spring-loaded catheter into the heart cavity in a simplified perspective view.

### EXAMPLE 1

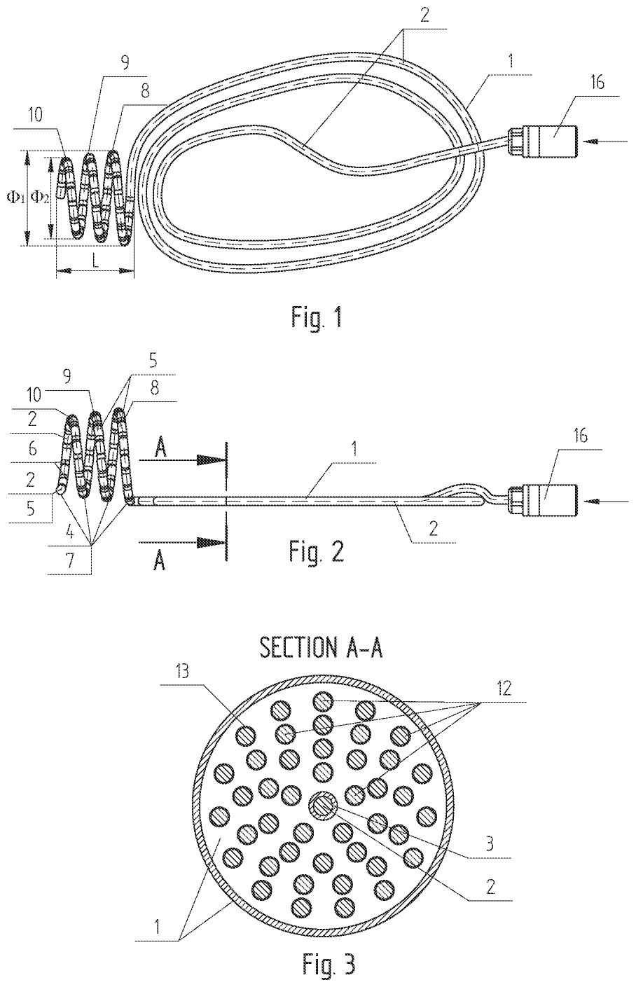

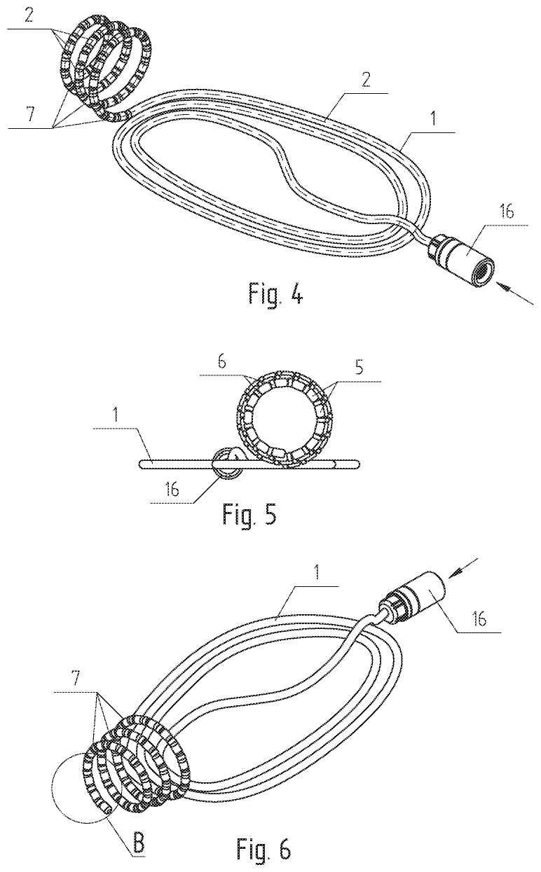

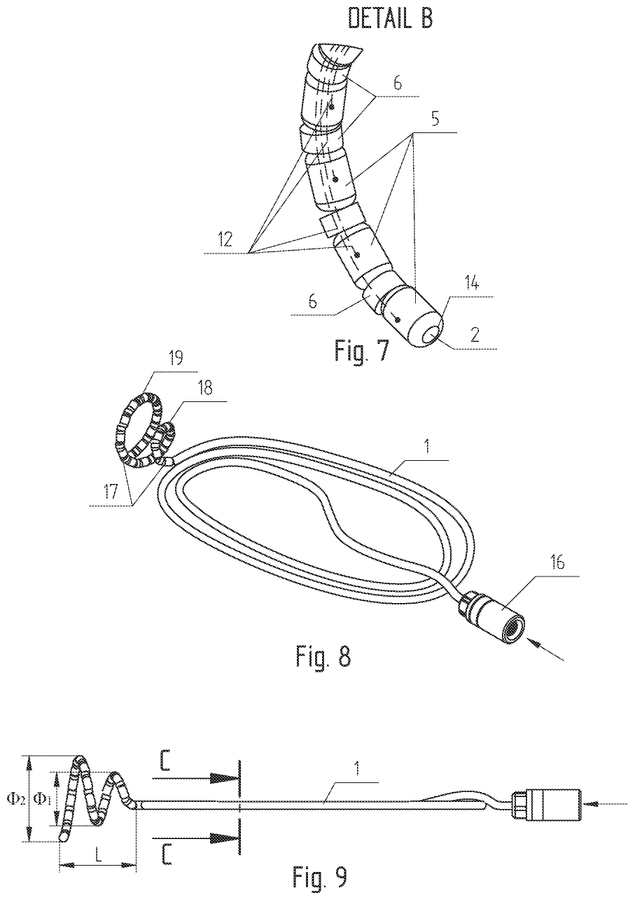

The spring-loaded catheter for electrophysiology studies and cardiac irreversible electroporation according to the first embodiment (FIGS. 1-7) is a plastic main conduit 1 made of a thermoplastic elastomer with a sleeve profile, with a core 2 made of nitinol (an alloy of metallic nickel with titanium showing shape memory effect) inside, covered with an insulating, plastic coating 3, where forty-three sleeve electrodes 5, separated by plastic non-conductive ring elements 6, are placed on the end 4 of the core 2 protruding from the main conduit 1.

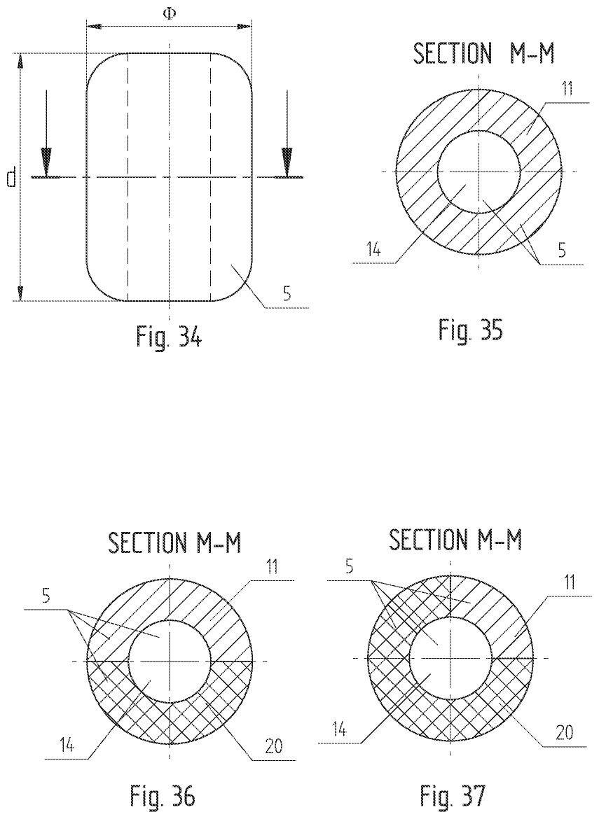

The end 4 of the core is bent in the shape of a conical spiral 7 with a length L=17 mm, forming three coils 8, 9 and 10, so that the first coil 8 has a diameter Ø1 equal to 30 mm, and the last coil 10 has a diameter Ø2 equal to 10 mm and finished with a sleeve electrode 5. Each of the electrodes 5 is entirely made of a homogeneous electrically conductive material 11—surgical steel and has a length d=2 mm and a diameter Ø=1 mm, as shown in FIGS. 34 and 35.

Inside the main conduit 1, between its inner surface and the outer surface of the cover 3 of the core 2, there are placed forty-three electric wires 12, made of copper with a diameter of 0.02 mm, surrounded and laminated with the sheath 13, the front ends of which are electrically connected to the corresponding sleeve electrodes 5, the end 4 of the core 2 and the electric wires 12 pass through the holes 14 of the electrodes 5 and the through holes 15 of the plastic annular elements 6 so that the core 2 passes through all the sleeve electrodes 5, while one electric conductor 12 is led to only one sleeve electrode 5.

In turn, the rear end of the main conduit 1 is electrically connected to a connector 16, for example of the Redel type, provided with forty pins, not shown, to which an electric current is supplied from an adapter also not shown providing high-amplitude electrical pulses, where the length of the entire of the spring-loaded catheter was 1.2 m.

### EXAMPLE 2

The spring-loaded catheter for an electrophysiology study and irreversible electroporation within the heart according to the second embodiment (FIGS. 8-10) is similar to the embodiment described in the first example, the difference being that in the latter embodiment, the front end 4 of the core 2 protruding from the main conduit 1 covered with an insulating, plastic coating 3 made of thermoplastic rubber is provided with twenty sleeve electrodes 5 separated by plastic ring elements 6 connected with twenty electric conductors 12 with a diameter of 0.2 mm, and it is bent into the shape of a conical divergent spiral 17 with length L=15 mm, forming two coils 18 and 19, with coil 18 having a diameter Ø1 equal to 5 mm and coil 19 having a diameter Ø2 equal to 31 mm. Moreover, in the embodiment, the electrodes 5 have a length d=4 mm, a diameter Ø=3 mm and are made of two materials such that one half of each is electrically conductive 11, platinum, and the other half is non-conductive PVC type as shown in FIGS. 34 and 36.

### EXAMPLE 3

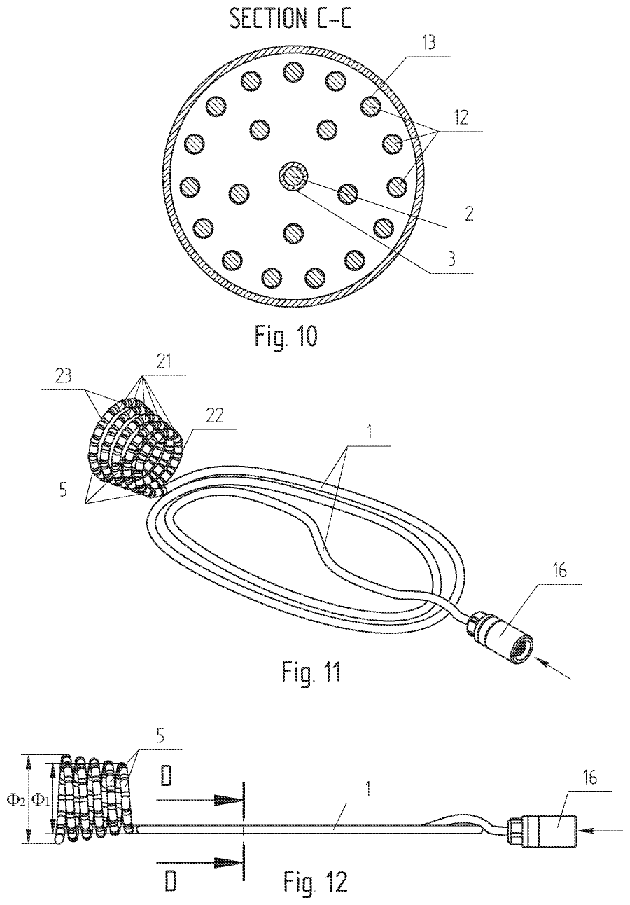

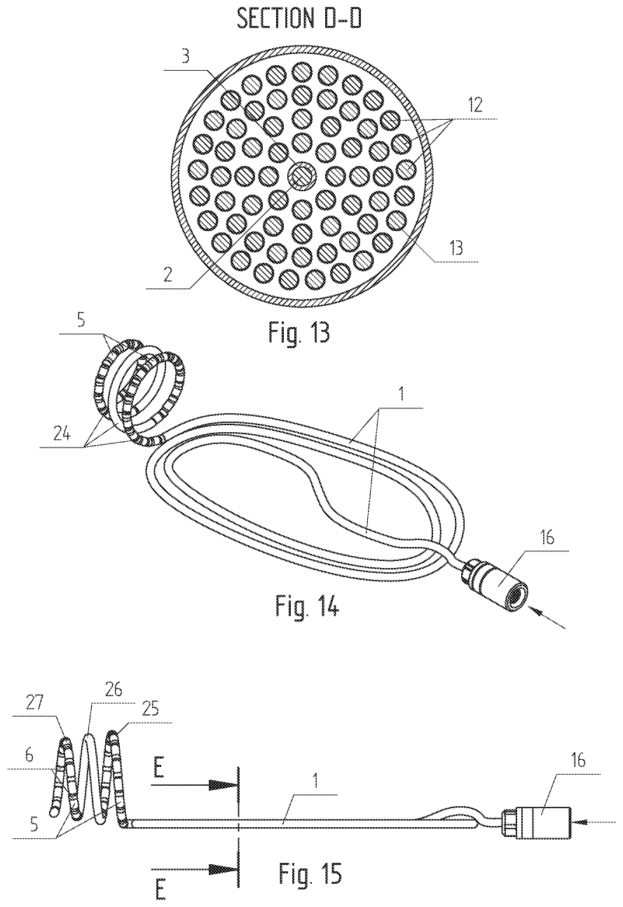

The spring-loaded catheter for an electrophysiology study and irreversible electroporation within the heart according to the third embodiment (FIGS. 11-13) is similar to the embodiment described in the first example, the difference being that in the third embodiment the front end 4 of the core 2 protruding from the main conduit 1 is provided with sixty-five sleeve electrodes 5, connected to sixty-five electric conductors 12, and is bent in the shape of a conical divergent spiral 21, forming five coils, the diameter Ø1 of the first coil 22 is 15 mm and the diameter Ø6 of the last coil 23 is 20 mm, wherein ¾ of the diameter of each of the sleeve electrodes 5 is non-conductive material 20—Teflon and ¼ of the electrically conductive material 11 is gold as shown in FIGS. 34 and 37, and the length of the entire spring-loaded catheter is 1.6 m.

### EXAMPLE 4

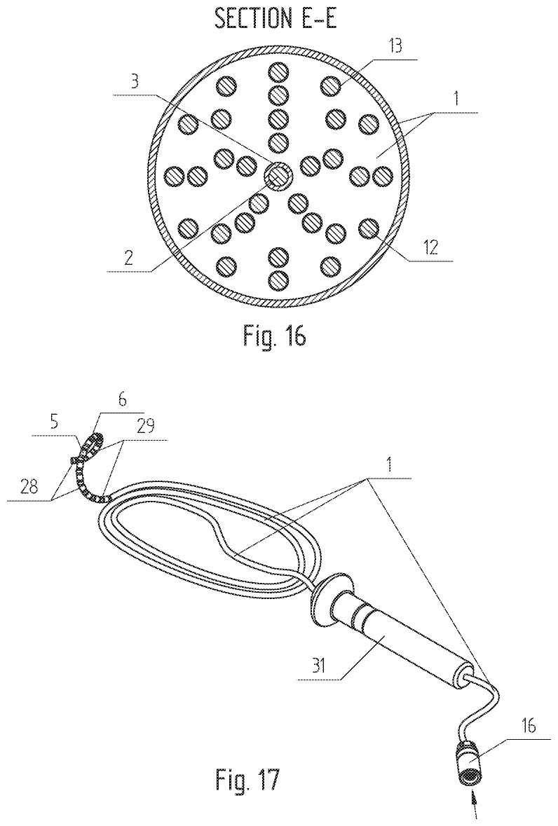

The spring-loaded catheter for an electrophysiology study and irreversible electroporation within the heart according to the fourth embodiment (FIGS. 14-16), it is similar to the embodiment described in the first embodiment, the difference between them being that in the fourth embodiment, the front end 4 of the core 2 protruding from the main conduit 1 is bent into the shape of the conical spiral 24, forming three coils 25, 26 and 27. Each of the coils 25 and 27 has fifteen sleeve electrodes 5 separated by plastic ring elements 6, and the coil 26, covered with a plastic coating 3, constitutes the core 2 with electric conductors 12 supplying electricity to sleeve electrodes 5 of coil 27.

### EXAMPLE 5

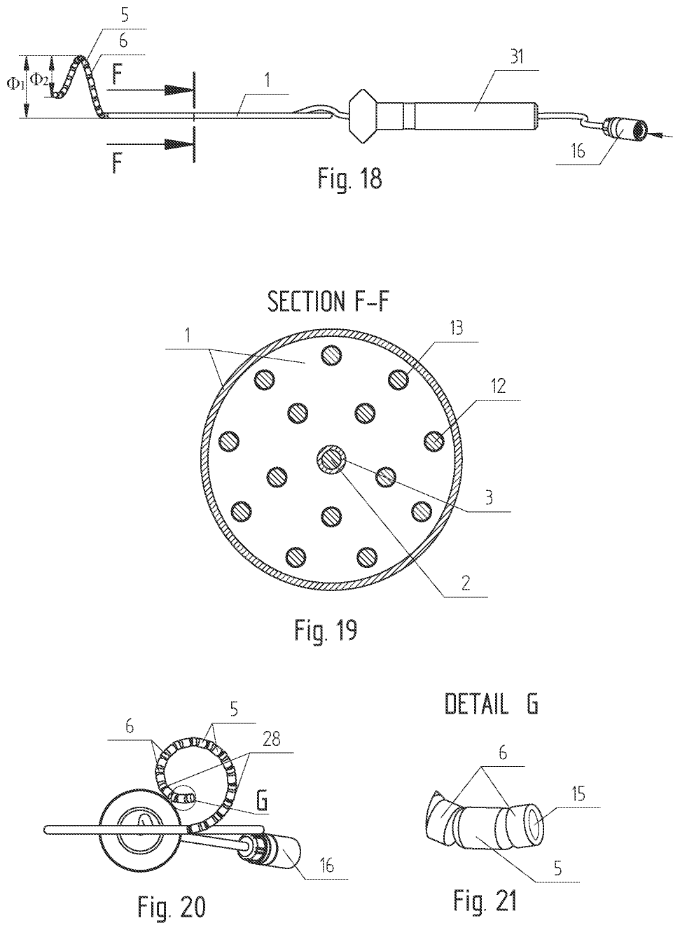

The spring-loaded catheter for an electrophysiology study and irreversible electroporation within the heart according to the fifth embodiment (FIG. 17-21), it is similar to the version of its embodiment described in the first example, the difference between them being that in the fifth embodiment, the front end 4 of the core 2 protruding from the body 1 is bent into a shape of the conical convergent spiral 28 forming one incomplete coil 29, formed by fourteen sleeve electrodes 5 connected by fourteen electric wires 12, equipped with thermistors 30 also connected by fourteen electric wires 12 with pins placed in connector 16, the initial diameter Ø1 of coil is 25 mm and the final diameter Ø2 is 10 mm, and the spiral 28 ends with an annular element 6. On the rear end of the sleeve main conduit 1, in front of the electrically connected connector 16, there is a driver handle 31, serving only to bend the end of the catheter spiral, which improves its steerability, the overall length of the catheter being 1.0 m.

In another embodiment of a fifth type catheter not shown on the figures, the number of sleeve electrodes 5 was ten.

### EXAMPLE 6

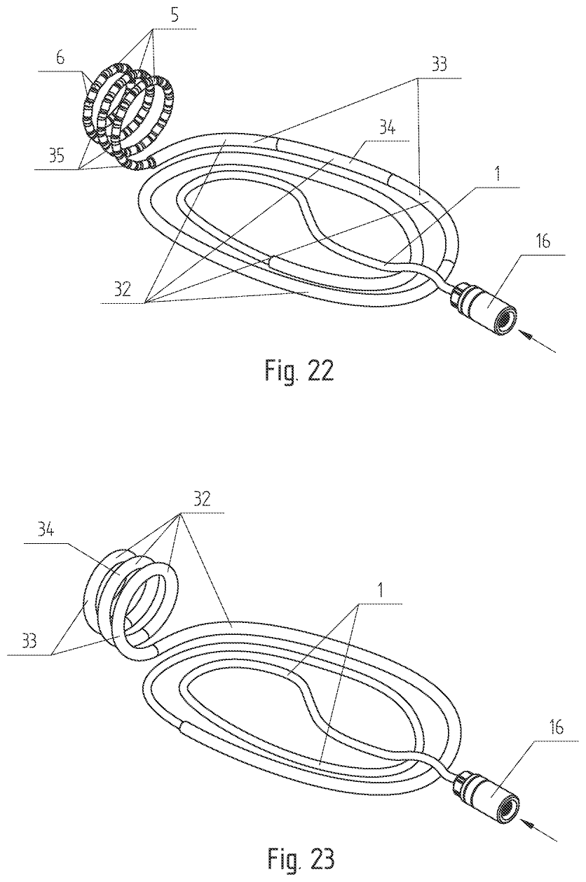

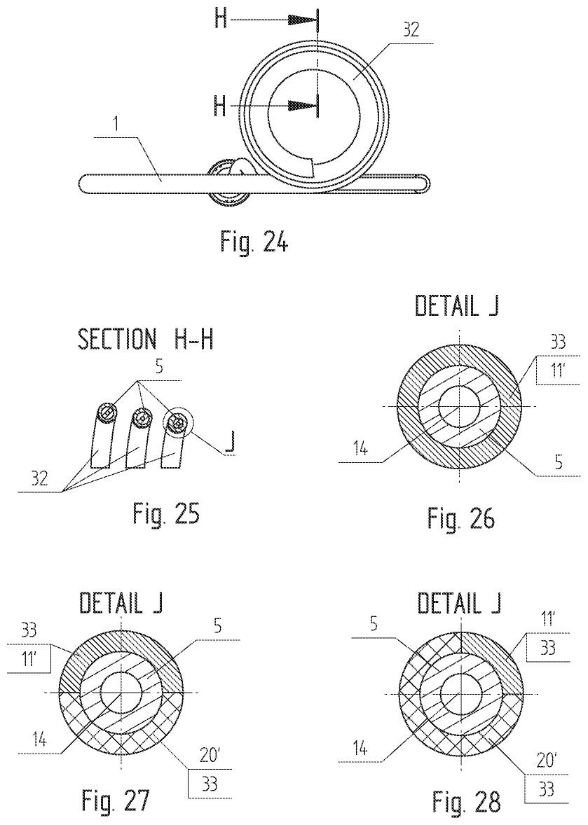

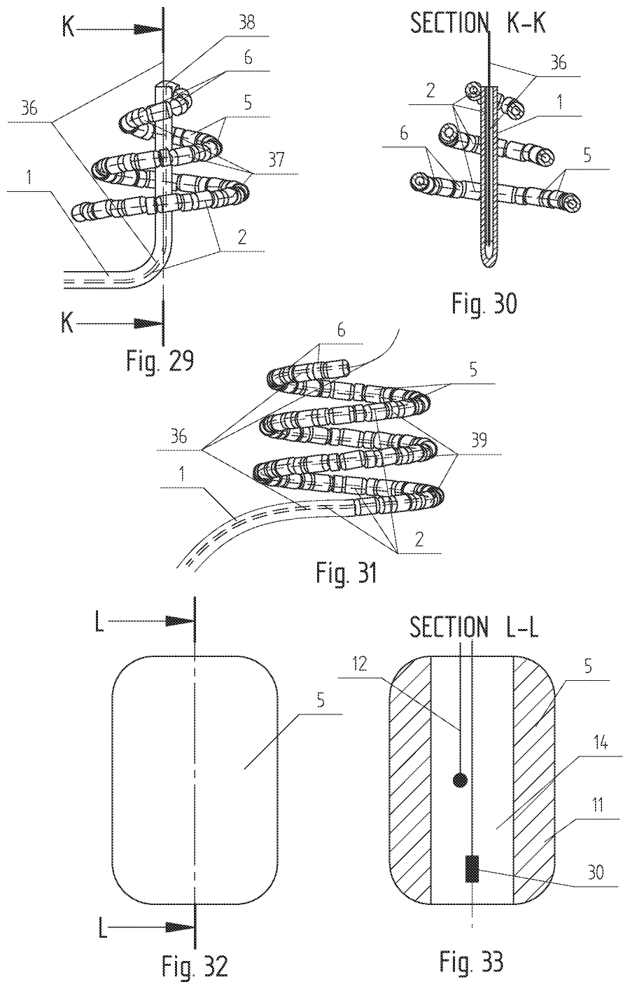

The spring-loaded catheter for an electrophysiology study and irreversible electroporation within the heart according to the sixth embodiment (FIGS. 22-28) is similar to the embodiment described in the first example, and the difference between them was that in the sixth embodiment, a sheath 32, which consists of electrically conductive sheaths 33 additionally mounted on the main sleeve conduit 1, made entirely of copper or its alloy (as shown in FIGS. 22 and 25) separated by an insulating plastic coating 34, with the end of sheath 32 is not electrically connected to prevent electric shock during operation of the catheter. When necessary, during treatment, sheath 32 slides over spiral-shaped sleeve electrodes 5 (as shown in FIG. 23), allowing a pulse to be transmitted between the two portions of the conductive sheath 33, increasing the effective surface area of the spring-loaded catheter.

In the embodiment of the conductive sheath 33 shown in FIG. 27, according to the sixth embodiment the conductive sheath 33 was made in half of electrically conductive material 11′ and in half of insulating material 20′, and in the example shown in FIG. 28 only ¼ of the sheath was electrically conductive 11′, and ¾ of electrically non-conducting insulating material 20′.

### EXAMPLE 7

The spring-loaded catheter for an electrophysiology study and irreversible electroporation within the heart according the seventh embodiment (FIGS. 29 and 30), is similar to the embodiment described in the first example, the difference between them being that in the seventh embodiment, a stabilizing rod 36 made of stainless steel is additionally placed inside the main conduit 1 covered with PTFE material, its conical spiral 37 is wound on the sleeve main conduit 1, and the stabilizing rod 36 extends from the main conduit 1 through the opening 38 and does not pass through sleeve electrodes 5 and plastic annular elements 6.

### EXAMPLE 8

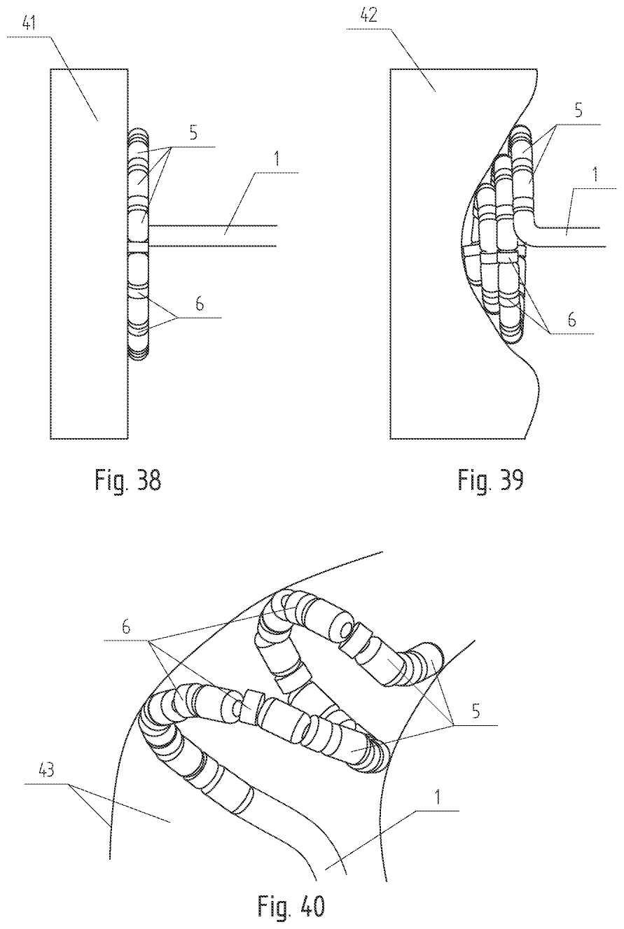

The spring-loaded catheter for an electrophysiology study and irreversible electroporation within the heart according to the eighth embodiment (FIG. 38) is similar to the embodiment described in the seventh embodiment, the difference between them is that in the eighth embodiment, the stabilizing rod 36 located in the sleeve main conduit 1 also passes through the sleeve electrodes 5 and plastic ring pieces of 6 conical spiral 39.

The additional stabilizing bar 36 described in Examples 7 and 8 is a stabilizing element for the spiral 38 and 39 allows the catheter according to the invention to access very narrow veins in the human heart.

In other embodiments of the spring-loaded catheter for an electrophysiology study and cardiac irreversible electroporation (not shown), the sleeve electrodes 5 had thermocouples embedded inside them, and the core 2 was made of shape memory metal alloys such as Cu—Al and Cu—Zn—Al alloys.

After the patient is prepared for electrophysiology study, puncture of the femoral vein, femoral artery, radial artery or brachial artery is performed, and through the puncture, using the Seldinger method, a venous or arterial sheath is inserted into the artery, 43 through which a spring-loaded catheter is inserted, the front of which is the part which in case of contact with the flat part of the heart surface 41 takes the form of a ring with coils arranged therein, or in case of the concave surface 42 takes the form of a corresponding cone as shown in FIGS. 38 and 39 adapted to the profile.

Signals from individual pairs of electrodes placed on the catheter are received and transmitted, depending on the need, to:
an electrophysiology system that enables imaging, recording and analysis of intracardiac potentials
a stimulator to deliver pulses that stimulate the heart to perform diagnostic manoeuvres
3D mapping system to reconstruct the catheter and/or the heart cavities
high amplitude pulse generator for electroporation or cardioversion/defibrillation

The electroporation process is usually carried out using a programmable generator with a voltage of 100-3000V, the pulse duration is from 5 microseconds to 6 milliseconds, while in case of using an automatic generator with a power of 5 J to 400 J.

What is claimed is:

1. A spring-loaded catheter for electrophysiology studies and irreversible electroporation within the heart has a plastic main conduit connected at one end to an electrical connector, from which electrodes located at the other end of the conduit are supplied via electric wires, characterized by the fact that the core protruding from the main sleeve conduit is made of a metal alloy that retains shape memory and is bent in the shape of a conical spiral with a different number of coils, at least one of which is equipped with overlapping on this core, sleeve electrodes fed through insulated electric wires and separated from each other by plastic ring elements, where the diameter Ø1 of the first coil of the spiral ranges from 5 mm to 30 mm, and the diameter Ø2 of the last coil of the spiral amounts to 10 mm to 31 mm, while the length of each of these electrodes is from 2 mm to 4 mm, and the diameter Ø is from 1 mm to 3 mm, where the electrodes which send a pulse with an amplitude of 100-3000V in time and from 5 microseconds to 6 milliseconds, and the number of electrodes located on the catheter spiral is from 10-65 pieces.

2. The spring-loaded catheter according to claim 1, characterized in that the conical spiral is a tapering spiral.

3. The spring-loaded catheter according to claim 1, characterized in that the conical spiral is a divergent spiral.

4. The spring-loaded catheter of claim 1, characterized in that the maximum number of coils of the spiral in the catheter is 5 coils and the number of sleeve electrodes arranged on the coils of the spiral is 65.

5. The spring-loaded catheter according to claim 1, characterized in that the two terminal coils of the conical spiral have 15 sleeve electrodes separated by plastic ring elements, and the central coil of the spiral is covered with a plastic sheath protecting the core together with electric wires supplying current to the sleeve electrodes of the coil.

6. The spring-loaded catheter according to claim 1, characterized in that three-part sheath is slidably placed on the sleeve main conduit, the two terminal parts of which are conductive sheaths, and the third sheath placed between them is made of insulating material, the conductive sheaths are made entirely of electrically conductive material or are in half made of electrically conductive material and in half of insulating material or ¼ of these sheaths are made of electrically conductive material, and ¾ of insulating material.

7. The spring-loaded catheter according to claim 6, characterized in that the electrically conductive material is copper or a copper alloy.

8. The spring-loaded catheter according to claim 1, characterized in that the stabilizing rod made of PTFE-coated stainless steel is inserted into the sleeve main conduit.

9. The spring-loaded catheter of claim 8, characterized in that the stabilizing rod exits the main conduit through the opening in front of the conical spiral so that the spiral is wound around the main conduit.

10. The spring-loaded catheter according to claim 8, characterized in that the stabilizing rod, placed in the sleeve main conduit, passes through the holes of the sleeve electrodes and the holes of the plastic ring elements of the conical spiral of the catheter.

11. The spring-loaded catheter according to claim 1, characterized in that it ends with a sleeve electrode.

12. The spring catheter according to claim 1, characterized in that it terminates with a plastic annular element.

13. The spring-loaded catheter according to claim 1, characterized in that at the rear end of the sleeve main conduit, a driver handle is provided in front of the electrically connected connector for bending only the end of the catheter spiral.

14. The spring-loaded catheter according to claim 1, characterized in that the sleeve electrodes are provided with thermistors.

15. The spring-loaded catheter according to claim 1, characterized in that the sleeve electrodes are provided with thermocouples.

16. The spring-loaded catheter according to claim 1, characterized in that the sleeve electrodes are entirely made of electrically conductive material.

17. The spring-loaded catheter according to claim 1, characterized in that half of the diameter of the sleeve electrodes are made of electrically conductive material and half of electrically non-conducting material.

18. The spring-loaded catheter according to claim 1, characterized in that the sleeve electrodes in ¼ of their diameters are made of electrically conductive material, and in other ¾ of non-conductive material.

19. The spring-loaded catheter according to claim 1, characterized in that the electrically conductive material of the sleeve electrodes is platinum, gold or surgical steel and the electrically non-conductive material is PVC or Teflon.

20. The spring-loaded catheter according to claim 1, characterized in that its core is made of nitinol and is covered with a plastic sheath.

21. The spring-loaded catheter according to claim 1, characterized in that the number of pins placed in the connector corresponds to the number of electric wires supplying the sleeve electrodes and the number of sensors placed in these electrodes.
